(19) United States
(12) Patent Application Publication
(10) Pub. No.: US 2002/0128293 A1
(43) Pub. Date: Sep. 12, 2002

(54) Stable oral pharmaceutical composition containing omeprazole

(76) Inventors: Ashok Rampal, Punjab (IN); Pratik Kumar, Bihar (IN); Girish Kumar Jain, Delhi (IN); Dilipkumar Thacharodi, (US)

Correspondence Address: Ranbaxy Pharmaceuticals Inc., Suite 2100, 600 College Road East, Princeton, NJ, 08540, US

(21) Appl. No.: 09/973,265

(22) Filed: Oct. 9, 2001

Related U.S. Application Data
(63) Continuation-in-part of application No. 09/086,224, filed on May 28, 1998, now abandoned.

(51) Int. Cl. A61K 31/4439

(52) U.S. Cl. 514/338

(57) ABSTRACT

The present invention relates to a stable oral pharmaceutical composition comprising omeprazole as the active ingredient and a carrier which acts as a stabilizing excipient. The invention also relates to a process for making the pharmaceutical composition.

### FIELD OF THE INVENTION

The present invention relates to a stable oral pharmaceutical composition comprising omeprazole as the active ingredient and a carrier which acts as a stabilizing excipient. The invention also relates to a process for making the pharmaceutical composition.

### BACKGROUND OF THE INVENTION

U.S. Pat. Nos. 4,255,431; 4,628,098; and 4,758,579 disclose substituted pyridylsulfinyl benzimidazoles (such as omeprazole) as potent inhibitors of gastric acid secretion. This class of compounds inhibits gastric acid secretion by inhibiting H₊-K₊ ATPase (proton pump) activity. Drugs in this class are known to be highly unstable in an acidic environment. They are also unstable in the presence of moisture and organic solvents. Thus, the formulation in which the drugs are to be administered to a patient, and the process for manufacture of the formulation, must be designed to protect the drug from moisture as well as an acidic environment. Due to the very rapid drug degradation which occurs in acidic gastric fluids, the formulations should also be enteric coated.

U.S. Pat. No. 4,786,505 discloses an oral pharmaceutical composition comprising a core containing omeprazole together with an alkaline reacting compound, or an alkaline salt of omeprazole optionally together with an alkaline compound; one or more subcoating layers comprising inert reacting compounds which are soluble or rapidly disintegrating in water, or polymeric, water soluble film forming compounds, optionally containing pH-buffering alkaline compounds; and an outer enteric coat. The alkaline reacting compound is a pharmaceutically acceptable substance (or substances) which creates a “micro-pH” around each omeprazole particle of not less than pH=7, preferably not less than pH=8, when water is adsorbed onto the particles of the mixture or when water is added in small amounts to the mixture. The subcoating layer separates the omeprazole containing core from the enteric coating polymer(s) containing free carboxyl groups. The enteric coating polymers can otherwise cause degradation of omeprazole during the coating process or during storage.

Japanese Patent 05-194,225 discloses tablets, granules and capsule formulations where the benzimidazole gastric ulcer inhibitors are stabilized by compounding with amino acids and buffering agents.

U.S. Pat. No. 5,385,739 discloses a stable microgranule formulation containing a neutral core of sugar and starch and an active layer consisting of a dilution of omeprazole in mannitol in substantially equal amounts, wherein the active omeprazole layer contains about 10% by weight of carboxymethylstarch, and about 5% by weight of sodium lauryl sulfate, and wherein the dilution of omeprazole in mannitol is applied to the neutral core by means of hydroxypropyl methylcellulose as a high viscosity binder.

WO 97/12581 discloses a composition comprising: (a) a core containing omeprazole as the active principle, the core being constituted of nuclei and the omeprazole active principle mixed together and then compressed together, the omeprazole active principle not being in the form of an alkaline salt; (b) an intermediate layer; and (c) an enteric layer. The composition disclosed therein is stated to be free of alkaline reacting compounds which had previously been considered as essential; however, each of the compositions exemplified in WO 97/12581 contains either a lubricant, such as sodium stearyl fumarate, magnesium stearate, or talc in the core, or talc in the intermediate layer. These compounds are alkali metal or alkaline earth metal salts and are known to be alkaline in nature.

WO 00/78284 claims a stable composition for benzimidazole derivatives, the composition comprising a substrate, said substrate featuring the benzimidazole derivative; and an enteric coating material layered directly over said substrate, said enteric coating material having a pH value of at least about 6.5, thereby obviating the need for an intermediate layer between said substrate and said enteric coating.

U.S. Pat. Nos. 6,096,340 and 6,077,541 disclose a pharmaceutical composition of omeprazole in pellet form wherein the pellet comprises an inert core comprising omeprazole, a surface active agent, a filler, a pharmaceutically acceptable alkaline agent and a binder; and an enteric coating layer.

WO 00/12064 makes use of basic amino acids lysine and arginine for stabilizing omperazole followed by enteric coating of the core without any intermediate separating layer.

U.S. Pat. No. 5,626,875 assigned to Esteve Quimica discloses compositions of omeprazole comprising a core composed of omeprazole and a non-alkaline inert water soluble polymer and excipients; an inert non-alkaline coating and an enteric coating.

Our co-pending PCT application WO 99/61022 discloses a stabilized formulation of omeprazole wherein the composition is stabilized with a polymer containing vinylpyrrolidone units.

### SUMMARY OF THE INVENTION

It is an object of the present invention to provide a stable oral pharmaceutical composition containing omeprazole as the active ingredient and a pharmaceutically acceptable carrier, which composition is free of alkaline compounds.

It has been surprisingly found that in a mixture comprising omeprazole and one or more polymers obtained by the polymerization of monomers at least one of which is vinylpyrrolidone, the omeprazole is stabilized. The mixture, even though it is free of alkaline reacting compounds, does not show a change in color which is typically observed in compositions where the omeprazole has undergone degradation.

Accordingly, the present invention provides a pharmaceutical composition which is stable and suitable for oral administration to a patient, comprising a mixture of omeprazole, and a pharmaceutically acceptable carrier, said carrier comprising at least one water insoluble polymer. The polymer is at least partially comprised of vinylpyrrolidone units. Optionally, the mixture also contains other pharmaceutically acceptable excipients. The composition may be in the form of a simple powder blend or granules of the active ingredient and the carrier, together with any optionally included excipients, filled into an enteric capsule, i.e., a capsule which is coated with an enteric polymer or which is made from an enteric polymer, or in the form of a bead or a pellet wherein said mixture of the active ingredient and the carrier, together with any optionally included excipients is coated on a neutral core or non-pariel seeds.

According to another aspect of the present invention, it provides a stable formulation of omerprazole in the form of a bead or a pellet, comprising (a) a neutral core coated with a mixture of omerprazole and a pharmaceutically acceptable carrier, said carrier comprising at least one water insoluble polymer, (b) one or more intermediate layer(s), optimally comprising water soluble or insoluble polymers, and (c) an enteric coating layer, which gives the desired rapid rate of dissolution and absorption of drug.

The present invention also provides a process for making a pharmaceutical composition which is stable and suitable for oral administration to a patient, comprising mixing omeprazole together with a pharmaceutically acceptable carrier, said carrier comprising at least one water insoluble polymer, together with any optionally included pharmaceutically acceptable excipients. The polymer is at least partially comprised of vinylpyrrolidone units. The mixture, which is in the form of a simple powder blend is then filled into enteric capsules, i.e., capsules which are coated with an enteric polymer or which are made from an enteric polymer. The mixture, in the form of a powder blend, may be converted into granules and the granules are filled into an enteric capsule.

Alternatively, the mixture may be coated on a neutral core or non-pariel seeds. Neutral core, may in turn, be previously coated with a coating mixture containing water soluble or insoluble polymers having vinylpyrrolidone units, together with optionally included pharmaceutically acceptable excipients, without the active ingredient, omeprazole. Neutral core coated with said mixture of the active ingredient and the carrier, may further be coated with one or more intermediate layers, and finally with an enteric coated layer. The enteric coated layer is the one which has an enteric polymer. Intermediate layer(s) may contain a water soluble or insoluble polymer together with optionally included pharmaceutically acceptable excipients.

### DETAILED DESCRIPTION OF THE INVENTION

According to the present invention, in addition to omeprazole, the pharmaceutical composition contains a carrier comprising one or more polymers that are obtained by polymerization of monomers at least one of which is vinylpyrrolidone.

An example of a class of polymers that may be used in the present invention is polyvinylpyrrolidones also known as povidone or PVP. The United States Pharmacopoeia XXII describes povidone as a synthetic polymer consisting essentially of linear 1-vinyl-2-pyrrolidone groups. The polyvinylpyrrolidones, are commonly available from BASF under the brand name Kollidon or from ISP under the brand name Plasdone. Polyvinylpyrrolidone is available as a water soluble polymer or as a cross-linked water insoluble polymer. Examples of water soluble polyvinylpyrrolidones, include PVP K-12, PVP K-15, PVP K-17, PVP K-25, PVP K-30, PVP K-60, PVP K-90, and PVP K-120 having approximate molecular weights of 2500, 8000, 10000, 30000, 50000, 400000, 1000000, and 3000000, respectively. Soluble PVP is conventionally used as a binder in tablet formulations. In the present invention, soluble PVP is used in the inventive composition as a stabilizing excipient and as a diluent for omeprazole.

Cross-linked polyvinylpyrrolidone is a polymer obtained by a polymerization process that produces a physically cross-linked polyvinylpyrrolidone which is insoluble in water and in all the usual solvents. Examples of cross-linked polyvinylpyrrolidones that may be used in the present invention include various grades such as those available from BASF under the brand names Kollidon CL, Crospovidone M, and Kollidon CL-M. Because of its high swelling ability, cross-linked polyvinylpyrrolidone is conventionally used as a disintegrant in tablets; however, in the present invention it is used as a stabilizing excipient and as a diluent for omeprazole.

Another example of a class of polymers that may be used in the present invention are water soluble vinylpyrrolidone-vinyl acetate copolymers that are formed by the copolymerization of vinylpyrrolidone and vinyl acetate. An example of a vinylpyrrolidone-vinyl acetate copolymer that may be used in the present invention is the copolymer available from BASF under the brand name Kollidon VA-64. In the present invention, the vinylpyrrolidone-vinyl acetate copolymer is used as a stabilizing excipient and as a diluent for omeprazole.

According to the present invention, the pharmaceutically acceptable carrier is present in an amount which is at least about 40% by weight of omeprazole.

According to the present invention, the pharmaceutical composition may also contain conventional pharmaceutically acceptable excipients. Pharmaceutical excipients well known in the pharmaceutical arts can be found listed in the Handbook of Pharmaceutical Excipients (Ed. A. Wade and P. J. Weller, The Pharmaceutical Press, London), in the U.S. FDA listing of inactive ingredients, and in other sources of pharmaceutical literature.

In one embodiment, the pharmaceutically acceptable excipients may comprise fatty acid glycerides. One example of fatty acid glycerides that may be used in the invention is a mixture of glycerides (e.g., mono-, di- and/or triglycerides) of long chain (e.g., C₁₂-C₁₈) fatty acids; for example, the range of products available under the brand name Gelucire (Gattefosse Corporation). Another example of fatty acid glycerides that may be used in the invention is a mixture of glycerides (e.g., triglycerides) of medium chain length (e.g., C₈-C₁₀) fatty acids; for example, the range of products available under the brand names Miglyol, Crodamol GTC/C, MCT oil, Neobee M5, AKOMED, Nesatol, and the like. The fatty acid glycerides included in the composition of this invention can also be in the form of vegetable oils, such as castor oil, hydrogenated castor oil, or hydrogenated vegetable glycerides, such as those available under the brand name Witepsol.

According to the process of the present invention, omeprazole, a carrier comprising one or more polymers comprising vinylpyrrolidone monomeric units, optionally together with pharmaceutically acceptable excipients, are mixed together to obtain a blend or granules, the blend or granules so obtained are filled into capsules, and the capsules are then enteric coated, or the blend or granules are filled into capsules having an enteric coating or made from an enteric material. In embodiments where one of the pharmaceutically acceptable excipients is a fatty acid glyceride, a liquid fatty acid glyceride is mixed with the other ingredients of the composition, or a solid fatty acid glyceride is first heated to above its melting point and the liquid obtained mixed with other ingredients of the composition to obtain granules.

The capsules used in the invention may be hard or soft capsules. The outer shell of the capsules may be composed of a film forming agent or agents, water and plasticizer. The shell may also contain coloring and opacifying agents. Examples of film forming agents which may be used in the capsule shell include gelatin, methylcellulose, hydroxypropyl methylcellulose, hydroxypropyl cellulose, and the like. When the shell is of a conventional type, e.g., it is made from gelatin, it is given an outer enteric coat. Alternatively, the capsules are enteric capsules wherein the shell itself is enteric in nature. The shell of enteric capsules may be made from one or more (film forming polymers at least one of which has an enteric nature. The composition of enteric capsules is a known art. For example, the shell may be made from a mixture of polymers such as gelatin or hydroxypropyl methylcellulose, and one or more enteric polymers, such as a polyacrylate enteric polymer, cellulose acetate phthalate, hydroxypropyl methyl cellulose acetate succinate, or cellulose acetate butyrate; or from a mixture of gelatin or hydroxypropyl methylcellulose and polyvinyl acetate phthalate; or from calcium alginate, and the like. The enteric polymers may be present as free acids or their salts. When the shell is of a conventional type, an outer enteric coating may be applied using known art. The coating composition may be aqueous or organic solvent based. The drying of the applied layers of a coating composition may be achieved by conventional means or by application of vacuum.

In another embodiment of the process of the present invention, omeprazole, a carrier comprising one or more vinylpyrrolidone polymers, optionally together with pharmaceutically acceptable excipients, are mixed together to obtain a powder blend, and the blend so obtained is subjected to conventional processing steps to obtain granules or tablets.

In yet another embodiment of the present invention omeprazole is mixed with cross-linked PVP and coated on non-pariel seeds with the help of conventionally used lubricants, plasticizers, fillers, and binders. This is followed by the application of intermediate layer(s) optionally water soluble or insoluble polymers comprising, a sugar or mixtures thereof, and finally the application of an enteric layer. The enteric coated pellets thus obtained may be compressed into tablets or filled into a capsule.

According to the present invention, the pharmaceutically acceptable excipients may comprise lubricants. Examples of lubricants that may be used in the present invention include, but are not limited to talc, magnesium stearate, calcium stearate, polyethylene glycol, sodium stearyl fumarate, and mixtures thereof.

Plasticizers of the present invention are selected from a group consisting of triethyl citrate, polyethylene glycol, and mixtures thereof.

Mixture may contain one or more of commonly used fillers selected from a group consisting of lactose, sucrose, mannitol, microcrystalline cellulose, and the like.

According to the present invention the pharmaceutically acceptable excipients may also include a binder. The binders commonly known to the pharmaceutically art may be used in the present invention. Examples of the binders are polyvinylpyrrolidone, starch, low viscosity grade hydroxypropyl methylcellulose, hydroxymethyl cellulose, hydroxyethyl cellulose, hydroxypropyl cellulose, and the like.

The present invention is further illustrated by the following non-limiting examples.

### EXAMPLE 1

Omeprazole and cross-linked polyvinylpyrrolidone in amounts as given in Table 1 were mixed together. The blend so obtained was filled into capsules.

TABLE 1

| | Weight |
|---|---|
| Ingredient | (mg/capsule) |
| Omeprazole | 20.00 |
| Cross-linked polyvinylpyrrolidone | 100.00 |
| (Kollidon CL-M) | |
| Total | 120.00 |

The capsules were enteric coated in a Freund Hi-coater to a weight build-up of 10% using the coating composition as given in Table 2.

TABLE 2

| Ingredient | Weight (g) |
|---|---|
| Eudragit L-100-55 | 100.00 |
| Sodium hydroxide | 1.40 |
| Titanium dioxide | 1.70 |
| Talc | 50.00 |
| Polyethylene glycol-300 | 20.00 |
| Water | 650.00 |

### EXAMPLE 2

Omeprazole and vinylpyrrolidone-vinyl acetate copolymer in amounts as given in Table 3 were mixed together. The blend so obtained was filled into capsules.

TABLE 3

| | Weight |
|---|---|
| Ingredient | (mg/capsule) |
| Omeprazole | 20.00 |
| Vinylpyrrolidone-vinyl acetate copolymer | 100.00 |
| (Kollidon VA-64) | |
| Total | 120.00 |

The capsules were enteric coated in a Freund Hi-coater to a weight build-up of 10% using the coating composition as given in Table 2.

### EXAMPLE 3

Omeprazole, vinylpyrrolidone-vinyl acetate copolymer, and cross-linked polyvinylpyrrolidone in amounts as given in Table 4 were mixed together. The blend was filled into capsules.

TABLE 4

| | Weight |
|---|---|
| Ingredient | (mg/capsule) |
| Omeprazole | 20.00 |
| Cross-linked polyvinylpyrrolidone | 50.00 |
| (Kollidon CL-M) | |
| Vvinylpyrrolidone-vinyl acetate | 50.00 |
| copolymer (Kollidon VA-64) | |
| Total | 120.00 |

The capsules were enteric coated in a Freund Hi-coater to a weight build-up of 10% using the coating composition as given in Table 2.

### EXAMPLE 4

Omeprazole and polyvinylpyrrolidone (PVP K 30) in the amounts as given in Table 5 were mixed together. The blend so obtained was filled into capsules.

TABLE 5

| | Weight |
|---|---|
| Ingredient | (mg/capsule) |
| Omeprazole | 20.00 |
| PVP K30 | 100.00 |
| Total | 120.00 |

The capsules were enteric coated in a Freund Hi-coater to a weight build-up of 10% using the coating composition as given in Table 2.

### EXAMPLE 5

Omeprazole and other ingredients in the amount as given in Table 6 were mixed together. The blend so obtained was filled into capsules.

TABLE 6

| | Weight |
|---|---|
| Ingredient | (mg/capsule) |
| Omeprazole | 20.00 |
| Kollidon CL-M | 50.00 |
| Avicel PH 112 | 50.00 |
| Total | 120.00 |

The capsules were enteric coated in a Freund Hi-coater to a weight build-up of 10%.

### EXAMPLE 6

Omeprazole, and Kollidon CL-M in amounts as given in Table 7 were mixed together. AKOMED R (Fatty acid glyceride composed of caprylic/capric triglycerides and derived from coconut and/or palm kernel oils) and Gelucire 33/01 (a mixture of glycerides e.g., mono-, di- and/or triglycerides of long chain fatty acids) were heated to 60° C. for 20 minutes, stirred well and cooled to 30° C. The omeprazole and the Kollidon blend were granulated with the liquid mix. The granules were screened through sieve no.22 and filled into capsules. The capsules were enteric coated in a Frenned Hi-coater to a weight build-up of 10%, using the coating composition as given in Table 2.

TABLE 7

| | Weight |
|---|---|
| Ingredient | (mg/capsule) |
| Omeprazole | 20.00 |
| Cross-linked polyvinylpyrrolidone | 100.00 |
| (Kollidon CL-M) | |
| Gelucire 33/01 | 10.00 |
| AKOMED R | 20.00 |
| Total | 150.00 |

The enteric coated capsules of examples 1 to 6 were tested as described under dissolution test (Method B) for delayed release (enteric coated) dosage forms in the United States Pharmacopoeia XXIII, page 1795. In the acid stage omeprazole was not released from the capsules. The data for percent released in the buffer stage is given in Table 8.

TABLE 8

| | MEAN CUMULATIVE PERCENT RELEASED | | | | | |
|---|---|---|---|---|---|---|
| TIME (MINUTES) | Ex. 1 | Ex. 2 | Ex. 3 | Ex. 4 | Ex. 5 | Ex. 6 |
| 20 | 6.50 | 21.60 | 58.40 | 14.70 | 1.70 | 72.20 |
| 30 | 46.70 | 39.50 | 83.66 | 30.00 | 4.80 | 102.00 |
| 45 | 91.50 | 60.10 | 95.60 | 75.00 | 84.90 | 106.80 |

the enteric coated capsules of examples 1 to 6 were kept in high density polyethylene bottles at 40° C./75% RH (Relative Humidity). The pharmaceutical compositions filled in the enteric capsules did not show any sign of instability such as change in colour or appearance as given in Table 9.

TABLE 9

| | OBSERVATION | |
|---|---|---|
| EXAMPLE No. | 15 days, 40° C./75% RH | 30 days, 40° C./75% RH |
| 1 | No Change | No Change |
| 2 | No Change | No Change |
| 3 | No Change | No Change |
| 4 | No Change | No Change |
| 5 | No Change | No Change |
| 6 | No Change | No Change |

Omeprazole content in the stored capsules stored as given above for a period of 30 days was determined by a stability indicating HPLC method. The results are given in Table 10.

TABLE 10

| EXAMPLE No. | Assay |
|---|---|
| 1 | 105.10 |
| 2 | 100.19 |
| 3 | 99.85 |
| 4 | 99.66 |
| 5 | 98.07 |
| 6 | 95.20 |

### EXAMPLE 7

Non pariel seeds were charged into a wurster fluid bed apparatus (Glatte® Rohm Pharma) coated with a seal coat followed by the application of the active layer containing omeprazole. This was followed by the application of the separating layer and finally an enteric coat. The composition of the enteric coat was the same described in Example 1.

The enteric coated pellets were filled into a capsule such that each capsule 40 mg omeprazole.

TABLE 11

| Ingredient | mg/capsule |
|---|---|
| Non Pariel Seeds | 145 |
| Seal Coat | |
| Crosslinked Polyvinylpyrrolidone | 1.98 |
| Polyvinylpyrrolidone K30 (binder) | 0.66 |
| Talc | 0.65 |
| Polyethylene glycol | 0.33 |
| Water | qs |
| Active Layer | |
| Crosslinked Polyvinylpyrrolidone | 25.2 |
| Polyvinylpyrrolidone K30 (binder) | 15 |
| Talc | 10 |
| Omeprazole | 40.8 |
| Pure Water | qs |
| Intermediate Layer | |
| Sugar | 6.28 |
| Crosslinked Polyvinylpyrrolidone | 2.1 |
| Water | qs |
| Enteric coat | |
| Eudragit L-1005 ® | 40 |

TABLE 12
The capsules were subjected to dissolution at pH 6.8 in a USP-II apparatus
at 100 rpm. As can be seen from Table 2 the drug was rapidly released
under these conditions.

| Time (minutes) | Percent drug released |
|---|---|
| 10 | 87 |
| 20 | 94 |
| 30 | 95 |

### EXAMPLE 8

Non pariel seeds were charged into a wurster fluid bed apparatus (Glatte® Rohm Pharma) coated with a seal coat followed by the application of the active layer containing omeprazole. This was followed by the application of the separating layer and finally an enteric coat. The composition of the enteric coat was the same described in Example 1.

The enteric coated pellets were filled into a capsule such that each capsule 40 mg omeprazole.

TABLE 13

| Ingredient | mg/capsule |
|---|---|
| Non Pariel Seeds | 156 |
| Seal Coat | |
| Crosslinked Polyvinylpyrrolidone | 2.13 |
| Polyvinylpyrrolidone K30 (binder) | 0.69 |
| Talc | 0.69 |
| Polethylene glycol | 0.36 |
| Water | Qs |
| Active Layer | |
| Crosslinked Polyvinylpyrrolidone | 25.2 |
| Polyvinylpyrrolidone K30 (binder) | 15 |
| Talc | 10 |
| Omeprazole | 40.8 |
| Pure Water | Qs |
| Intermediate Layer | |
| Mannitol | 9.74 |
| Talc | 2.4 |
| Water | Qs |
| Enteric Layer | |
| Eudragit L-100-55 ® | 40 |

TABLE 14
The capsules were subjected to dissolution at pH 6.8 in a USP-II apparatus
at 100 rpm. As can be seen from Table 2 the drug was rapidly released
under these conditions.

| Time (minutes) | Percent drug released |
|---|---|
| 10 | 98 |

While the invention has been described by reference to specific embodiments, this was for purposes of illustration only. Numerous alternative embodiments will be apparent to those skilled in the art and are considered to be within the scope of the claimed invention.

While the present invention has been described in terms of its specific embodiments, certain modifications and equivalents will be apparent to those skilled in the art and are intended to be included within the scope of the present invention.

1. A pharmaceutical composition which is stable and suitable for oral administration to a patient, comprising a mixture of omeprazole and a pharmaceutically acceptable carrier, said carrier comprising at least one water insoluble polymer.

2. The composition of claim 1 wherein said polymer has vinylpyrrolidone units.

3. The composition of claim 2 wherein said polymer is cross-linked polyvinylpyrrolidone.

4. The composition of claim 1 wherein said carrier further comprises water soluble polymers.

5. The composition of claim 4 wherein said polymer is polyvinyl pyrrolidone or vinylpyrrolidone-vinyl acetate copolymer.

6. The composition of claim 1 wherein said carrier comprises at least about 40% by weight of omeprazole.

7. The composition of claim 1 wherein said mixture further comprises other pharmaceutically acceptable excipients.

8. The composition of claim 8 wherein said mixture further comprises a fatty acid glyceride.

9. The composition of claim 8 wherein said mixture further comprises lubricants, plasticizers, fillers and binders.

10. The composition of claim 10 wherein the lubricants are selected from the group consisting of talc, magnesium stearate, calcium stearate, polyethylene glycol, sodium stearyl fumarate, and mixtures thereof.

11. The composition of claim 10 wherein the plasticizers are selected from the group consisting of triethyl citrate, polyethylene glycol, and mixtures thereof.

12. The composition of claim 10 wherein the binder is selected from the group consisting of polyvinyl pyrrolidone, starch, low viscosity grade hydroxypropyl methylcellulose, hydroxymethyl cellulose, hydroxyethyl cellulose, hydroxypropyl cellulose, and mixtures thereof.

13. The composition of claim 10 wherein the fillers are selected from the group consisting of lactose, sucrose, mannitol, and microcrystalline cellulose.

14. The composition of claim 1 in the form of a capsule, said mixture being contained within a capsule shell made from an enteric material.

15. The composition of claim 14 wherein said mixture contained within said capsule shell is in the form of a powder blend.

16. The composition of claim 14 wherein said mixture contained within said capsule shell is in the form of granules.

17. The composition of claim 1 in the form of a capsule, said mixture being contained within a capsule shell which is coated with an enteric material.

18. The composition of claim 17 wherein said mixture contained within said capsule shell is in the form of a powder blend.

19. The composition of claim 17 wherein said mixture contained within said capsule shell is in the form of granules.

20. The composition of claim 1 in the form of a tablet.

21. The composition of claim 1 in the form of a bead or a pellet, wherein said mixture is coated on a neutral core.

22. The composition of claim 21, wherein said neutral core has previously been coated with a coating mixture before coating with said mixture of claim 1.

23. The composition of claim 22 wherein said coating mixture may contain water soluble or water insoluble polymers optionally with other pharmaceutically acceptable excipients.

24. The composition of claim 21 wherein neutral core coated with said mixture is further coated with one or more intermediate layers, and an outer enteric layer.

25. The composition of claim 24 wherein an enteric layer has an enteric polymer.

26. The composition of claim 24 wherein the intermediate layer(s) may contain water soluble or water insoluble polymers, optionally with other pharmaceutically acceptable excipients.

27. A pharmaceutical composition which is stable and suitable for oral administration to a patient, comprising (a) a neutral core coated with a mixture of omeprazole and a pharmaceutically acceptable carrier, said carrier comprising at least one water insoluble polymer, (b) one or more intermediate layer(s), optionally comprising water soluble or insoluble polymers, and (c) an enteric coated layer.

28. The composition of claim 27 in the form of a bead or a pellet.

29. The composition of claim 28 wherein the beads or pellets are compressed into tablets or filled in a capsule.

30. A process for preparing a stable pharmaceutical composition which is suitable for oral administration, comprising mixing omeprazole together with a pharmaceutically acceptable carrier, said carrier comprising at least one water insoluble polymer.

31. The process of claim 30 wherein said polymer has vinylpryrrolidone units.

32. The process of claim 31 wherein said polymer is cross-linked polyvinylpyrrolidone.

33. The process of claim 30 wherein said carrier further comprises water soluble polymers.

34. The process of claim 33 wherein said polymer is polyvinyl pyrrolidone or vinylpyrrolidone-vinylacetate copolymer.

35. The process of claim 30 further comprising mixing at least one pharmaceutically acceptable excipient into said mixture.

36. The process of claim 35 wherein said pharmaceutically acceptable excipient is a fatty acid glyceride.

37. The process of claim 35 wherein said pharmaceutically acceptable excipient is a lubricant, plasticizer, filler, or a binder.

38. The process of claim 30 further comprising filling said mixture in the form of a powder blend into a capsule made from an enteric material.

39. The process of claim 30 further comprising coating a capsule shell with an enteric material, and filling said mixture in the form of a powder blend into said capsule shell.

40. The process of claim 30 further comprising granulating said mixture to produce granules, and filling said granules into a capsule shell made from an enteric material.

41. The process of claim 30 further comprising granulating said mixture so as to form granules, coating a capsule shell with an enteric material, and filing said granules into said capsule shell.

42. The process of claim 30 further comprising compressing said mixture into tablets.

43. The process of claim 30 comprising coating said mixture on a neutral core.

44. The process of claim 31 wherein said neutral core has previously been coated before coating of said mixture.

45. The process of claim 30 wherein neutral core coated with said mixture is further coated with one or more intermediate layers, and an outer enteric layer.

46. The process of claim 30 further comprising forming beads or pellets.

47. The process of claim 46 further comprising compressing beads or pellets into tablets or filling said beads or pellets in a capsule.
